(19) United States
(12) Patent Application Publication
(10) Pub. No.: US 2021/0346162 A1
(43) Pub. Date: Nov. 11, 2021

(54) JOINT PROSTHESIS COMPONENT, RELATED SURGICAL INSTRUMENTATION FOR THE BONE PROCESSING AND PROSTHESIS MANUFACTURING METHOD

(71) Applicant: Limacorporate S.p.A., Villanova di San Daniele del Friuli (UD) (IT)

(72) Inventors: Fausto Sbaiz, Codroipo (UT) (IT); Matteo Boccalon, Bicinicco (UD) (IT)

(73) Assignee: Limacorporate S.p.A., Villanova di San Daniele del Friuli (UD) (IT)

(21) Appl. No.: 17/283,882

(22) PCT Filed: Oct. 10, 2019
(86) PCT No.: PCT/EP2019/077439

§ 371 Date: Apr. 8, 2021

(30) Foreign Application Priority Data
Oct. 12, 2018 (IT) 102018000009413

(51) Int. Cl. A61F 2/34; A61F 2/30; A61F 2/36

(52) U.S. Cl. CPC A61F 2/34; A61F 2/3094; A61F 2002/30461; A61F 2/30749; A61F 2002/343; A61F 2/3609

(57) ABSTRACT

The invention relates to a joint prosthesis component (200) adapted to be fixed to a first bone extremity (250) of a joint of a single patient having compromised anatomy, said joint prosthesis component (200) comprises: —at least one fastening portion (202, 205, 206, 207) adapted to be in contact with the first bone extremity; —at least one joint portion (4) adapted to be directly coupled with second bone extremity of the joint or with a conjugate second joint prosthesis component, in turn fixed to the second bone extremity. Advantageously, the fastening portion is at least partially customized to be specifically shaped with respect to the morphology of the first bone extremity processed by means of at least one surgical instrumentation.

### FIELD OF APPLICATION

The present invention relates to a joint prosthesis component adapted to be fixed to a first bone extremity of a joint of a single patient, in particular a first bone extremity having compromised anatomy.

The invention also relates to a surgical instrumentation for preparing the bone for the implant of the joint prosthesis component and a related method for manufacturing said component.

The invention finds useful application in the field of joint prostheses adapted to be implanted in patients with particularly compromised joint anatomy, for instance in case of prosthetic revision, and in particular in case of coxal anchor of hip prosthesis and ulnar component of elbow prosthesis.

The following description is made with non-limiting reference to the latter prosthetic components, even though this does not prevent from applying the invention to other components of hip or knee prosthesis or of other joint prostheses.

### PRIOR ART

As it is well known in this technical field, under particular pathological conditions of the joints it is recommended to intervene with the implant of a prosthesis. In particular, an arthroplasty surgery is indicated in case of serious damage to the joint surfaces due for instance to degenerative diseases such as rheumatoid arthritis and arthrosis or to fractures.

In order to pursue a successful prosthetic implant, the design of the prosthetic components, the surgical instruments used for the implant and the surgical technique must be aimed at pursuing a suitable primary and secondary stability and, on the one hand, at restoring the best possible biomechanical condition.

In particular, in order to ensure a suitable stability of the implant, it is advisable to maximize the filling of the existing bone defects and the bone-implant contact surface, so as to ensure a stable and long-lasting fastening and a suitable transfer of the loads as uniformly and anatomically as possible, all this by means of a surgical technique that is the least invasive possible.

The technical solutions nowadays adopted substantially provide for the alternative use of two types of joint prostheses: prostheses with standard components and prostheses customized based on the anatomy of the single patient.

Though advantageous under various aspects, and substantially fulfilling the purpose, both the standard prostheses and the customized prostheses do not allow obtaining a suitable stability and/or a correct joint biomechanics in patients with a particularly compromised joint anatomy, namely in the presence of a marked reabsorption of bone tissue, deformation and/or fracturing. This anatomic condition may in particular occur in patients who need a joint prosthesis revision surgery.

In particular, the joint prostheses of the standard type provide a finite number of prosthetic components and surgical instruments having predefined shapes and size. Therefore, the surgeon must choose the components and instruments that best suit the case. However, often the operator does not have the needed instruments and/or components and must find a compromise. As a skilled person is well aware of, in case of particularly altered joint bone anatomies, it is very complicated to find an optimal compromise among the available elements and components. One often finds himself in the condition of having to remove too much or too little bone tissue to try to match and stabilize one of the standard components to the bone, however having to accept a non-optimal positioning in the space of the component or a non-optimal biomechanical recovery, or to pursue the suitable positioning in the space of the component, accepting to lose in implant stability. Therefore, this approach ensures an optimal success of the implant only in patients having a joint anatomy which little differs from the physiological condition.

An alternative solution used in patients having compromised anatomy is described for example in patent application No. WO 2015/187038 A1 which relates to the use of a prosthesis which may be defined of the customized type. In other words, prosthetic components are customized in the design step, so that, once implanted, they perfectly match with the bone anatomy of the single patient. This solution was conceived to avoid the need of removing bone tissue to adapt with the implant in order to obtain an optimal contact area with the bone and restoration of biomechanical parameters, since it is the implant itself that already has a morphology matching with the bone anatomy of the single patient.

However, though advantageous under various aspects and substantially fulfilling the purpose, in case of particularly compromised anatomies this solution has some drawbacks. In particular, not always is it possible to design a prosthetic component such as to fill all defects of the bone tissue, to suitably adhere to the bone and to be positioned so as to ensure an optimal joint biomechanics.

Finally, a customized prosthetic component often needs the use of a surgical technique that is different, potentially more invasive, than those commonly used by surgeons to implant components of the standard type. In some extreme cases the customized prosthetic component would need a so complex and invasive surgical procedure that it cannot be performed, or the component would significantly lose its anatomical filling feature to be inserted into the anatomy.

The object of the present invention is to provide a joint prosthetic component adapted to be fixed to the compromised anatomy of a single patient, having structural and functional features such as to overcome the above drawbacks with respect to the prior art and to maximize the chances of success of the implant, thus ensuring optimal primary and secondary stability and joint biomechanics for each patient.

### SUMMARY OF THE INVENTION

The solution idea underlying the present invention is to conceive a joint prosthesis component capable of customization with respect to the compromised anatomy of the corresponding joint bone extremity of a single patient, such as to match with the suitably processed bone in order to obtain an optimal fixing and positioning of the prosthetic component.

Based on this solution idea, the previously identified technical problem is solved by a joint prosthesis component according to claim 1.

Specific embodiments of the joint prosthesis component are defined in the dependent claims.

The above joint prosthesis component is designed so as to optimize the morphology of the component itself in connection with a processing of the bone extremity which is predefined to contribute obtaining a successful implant for each patient with compromised joint anatomy.

The above component is customized for each single compromised anatomy of a patient in order to have a morphology defined based on a specific processing of the bone anatomy to maximize the bone-implant contact by filling the bone defects and obtain a suitable stability of the implant, meanwhile positioning the prosthetic component in such a way as to ensure an optimal biomechanics of the prostheses, all this implantable through a surgical technique as less invasive as possible.

In other words, conversely to the known prosthetic components, the positioning of the component and restore of the biomechanical parameters are not conditioned by the availability of finished prosthetic components or by the need of pursuing the anatomy or stability of the implant, the morphology of the component may be advantageously defined along with the optimal bone processing, so as to obtain a suitable positioning and stability of the implant for each patient with compromised anatomy.

The above processing may be advantageously performed with at least one surgical instrument, which may be in turn advantageously customized to make the specific processing aimed at preparing the bone to receive the corresponding component.

Furthermore, advantageously, the prosthesis component may provide for at least one porous trabecular three-dimensional structure and/or at least one suture hole to increase the integration of the implant with the bone and surrounding soft tissues.

Advantageously, the porous trabecular structure may be in turn customized for instance in terms of shape, positioning in the component, thickness, porosity, trabecular structure to adapt to the possibly processed compromised bone anatomy of the single patient. Furthermore, the positioning and size of the suture holes in the joint prosthesis component may also be customized according to the bone anatomy.

For instance, the suture holes may advantageously be formed at the porous three-dimensional structure in order to obtain in a determined area of the implant a synergic effect in promoting the integration with the surrounding tissues.

The joint prosthesis component may be a coxal anchor of a hip prosthesis in which the customized portion is represented by a distal surface of an acetabular support which adapts to the morphology of the acetabular cavity of the patient with a specifically processed compromised anatomy.

The coxal anchor may also comprise additional components to the acetabular support, which may be flanges, stems, wedges, thicknesses or inserts, that can be fixed to one or more of the anatomical portions that delimit the acetabular cavity—ileum, pubis and ischium—by cementing and/or by means of fixing screws.

The additional support provided by the additional components also allows fixing the coxal anchor to anatomical sites of smaller size with respect to the acetabular cavity, thus increasing the implant stability.

As previously mentioned, these additional components may advantageously be customized, so as to be shaped to the corresponding bone portion, also possibly processed by means of at least one surgical instrument, which in turn may advantageously be customized to perform the processing aimed at preparing the bone to receive the corresponding component.

The additional components may be integral with the acetabular support or with the latter in the implant step by cement interposition.

Alternatively, the coxal anchor may provide for a plurality of additional components, for instance flanges, having different size and/or morphological features, among which the surgeon may choose the most suitable ones to be used.

The joint prosthesis component may be an ulnar component of elbow prosthesis.

Said ulnar component may comprise a proximal fastening portion adapted to be fixed to a proximal epiphysis of the ulna of a patient with compromised anatomy and a distal fastening portion adapted to be fixed within a diaphysis of said ulna, wherein at least one of said proximal and distal fastening portions has a specific shape with respect to the morphology of a proximal portion of the ulna specifically processed by means of at least one surgical instrument.

The above identified technical problem is also solved by a surgical instrument for processing a first bone extremity of a patient with compromised anatomy aimed at implanting a joint prosthesis component according to what has been previously discussed, at least partially specifically shaped to process the first bone extremity so as to adapt to the customized surface of the joint prosthesis component.

Said surgical instrumentation may advantageously provide for at least one customized portion specifically shaped to process the bone so as to perfectly adapt to the joint prosthesis component. For instance, said customized processing portion may be specifically shaped to process the acetabular cavity of the coxal bone and/or ileum and/or pubis and/or ischium, so as to match with the distal surface of the acetabular support or with a corresponding additional component, respectively.

The above identified technical problem is also solved by a manufacturing method for manufacturing a joint prosthesis component adapted to be fixed to a first bone extremity of a joint of a single patient having compromised anatomy, comprising the steps of:
providing a joint prosthesis component having at least one bone fastening portion comprising at least one contact surface adapted to be in contact with the first bone extremity, and at least one joint portion adapted to be directly coupled to a second joint bone extremity or with a conjugate second joint prosthesis component, in turn fixed to the second extremity;
predefining at least one processing of first bone extremity that can be performed by means of at least one surgical instrument; this processing consisting for instance in removing bone tissue;
predefining the customization of at least one contact surface specifically shaped with respect to the morphology of said first bone extremity;
providing at least one customized contact surface in said joint prosthesis component.

The above manufacturing method may also provide for an optimization step of the processing of the first bone extremity and of the customization of the customized contact surface in the joint prosthesis component.

As a skilled person may well understand, the optimization of the bone processing and of the component morphology may advantageously be performed upstream of the surgery in order to identify the best possible combination of the two variables in connection with the compromised anatomy of the single patient.

The above manufacturing method may further include the step of providing at least one surgical instrumentation having at least one customized portion specifically shaped so as to perform the processing of the first bone extremity that adapts to the customized surface of the joint prosthesis component.

The features and advantages of the joint prosthesis component, of the instrumentation and of the manufacturing method according to the present invention will become apparent from the following description, of more than one possible exemplifying embodiment, given by way of non-limiting example with reference to the appended drawings.

### BRIEF DESCRIPTION OF THE DRAWINGS

FIG. 1 shows a perspective view of a first model of coxal bone having compromised anatomy with the cutting surfaces CS overlapped;

FIG. 2 shows a perspective view of the model of FIG. 1 with another cutting surface CS overlapped;

FIG. 3 shows the model of FIGS. 1 and 2 with a first example of coxal anchor implanted according to the invention;

FIG. 4 shows a perspective view of a second model of coxal bone with compromised anatomy with a second example of coxal anchor implanted according to the invention;

FIG. 5 shows a perspective view of a third model of coxal bone having compromised anatomy with a third example of coxal anchor implanted according to the invention;

FIG. 6 shows a perspective view of a fourth model of coxal bone having compromised anatomy with a fourth example of coxal anchor implanted according to the invention;

FIG. 7 shows another perspective view of the model of FIG. 5 with the fourth example of coxal anchor implanted according to the invention;

FIG. 8 shows a section view of the model of FIG. 5 with the fourth example of coxal anchor implanted according to the invention;

FIG. 9 shows a further perspective view of the model of FIG. 5 with the fourth example of coxal anchor implanted according to the invention;

FIG. 10 shows a perspective view of a fifth model of coxal bone having compromised anatomy with a fifth example of coxal anchor implanted according to the invention;

FIG. 11 shows a perspective view of a sixth model of coxal bone having compromised anatomy with a sixth example of coxal anchor implanted according to the invention;

FIG. 12 shows a perspective view of a seventh model of coxal bone having compromised anatomy with a seventh example of coxal anchor implanted according to the invention;

FIG. 13 shows a perspective view of an eighth example of coxal anchor according to the invention;

FIG. 14 shows a perspective view of an eighth model of coxal bone having compromised anatomy with a ninth example of coxal anchor implanted according to the invention;

FIG. 15 shows a side view of a first model of ulna having compromised anatomy with a first example of ulnar component implanted according to the invention;

FIG. 16 shows a perspective view of a processing instrumentation during the processing of the intramedullary cavity of the ulna of the model of FIG. 15;

FIG. 17 shows a further side view of the model of FIG. 15 with a first example of ulnar component implanted according to the invention;

FIG. 18 shows a detail of FIG. 17;

FIG. 19 shows a perspective view of a second model of ulna having compromised anatomy with a second example of ulnar component implanted according to the invention;

FIG. 20 shows a side view of a third example of ulnar component according to the invention;

FIG. 21 shows a perspective view of the ulnar component of FIG. 20;

FIG. 22 shows a perspective view of a total elbow prosthesis of the hinged type according to the prior art;

FIG. 23 shows a perspective view of a total elbow prosthesis of the non-hinged type according to the prior art.

### DETAILED DESCRIPTION

The present invention relates to a joint prosthesis component 10, 100, 200, 300, 400, 500, 600, 700, 800, 900, 1000, 1100, adapted to be fixed to a first bone extremity 50, 150, 250, 350, 450, 550, 650, 750, 850, 950, 1050 of a joint of a single patient, in particular a first bone extremity having compromised anatomy. “Single patient” obviously means a single and customized use of the prosthesis component.

The above component comprises at least one joint portion 4, 904 adapted to be directly coupled with a second bone extremity or with a conjugate second joint prosthesis component, in turn fixed to the second bone extremity.

The joint prosthesis component comprises at least one bone fastening portion 12, 102, 202, 302, 402, 502, 602, 702, 802, 902, in which at least one contact surface 13, 103, 203, 303, 403, 503, 603, 703, 803, 903 is defined, which is adapted to be directly in contact with the first bone extremity 50, 150, 250, 350, 450, 550, 650, 750, 850, 950, 1050, 1150.

Said contact surface advantageously has a morphology such as to perfectly match with the first bone extremity 50, 150, 250, 350, 450, 550, 650, 750, 850, 950, 1050, 1150 which has been subjected to a specific processing, for instance a bone tissue removal, by means of at least one surgical instrumentation 2000.

This processing may advantageously be performed by means of at least one surgical instrumentation having at least one customized processing portion to perform the desired specific processing. Obviously, this does not exclude that in some cases the processing may be performed by means of instruments having standard size and shape.

As it will become apparent from the examples which will be hereinafter discussed, a prosthetic component of this type may be designed considering two variables such as component morphology and bone processing, so as to conceive a prosthetic component which may obtain optimal stability and biomechanics of the prosthesis once implanted.

This solution may be an advantageous alternative to the solutions known in the field, namely to the use of standard or completely customized joint prostheses, in particular in case of a particularly compromised joint bone anatomy.

Hereinafter, with reference to the appended figures, examples of components of joint prosthesis according to the invention, namely of coxal anchor of hip prosthesis 10, 100, 200, 300, 400, 500, 600, 700, 800 and ulnar component of elbow prosthesis 900, 1000, 1100, will de deepened without any limiting purpose.

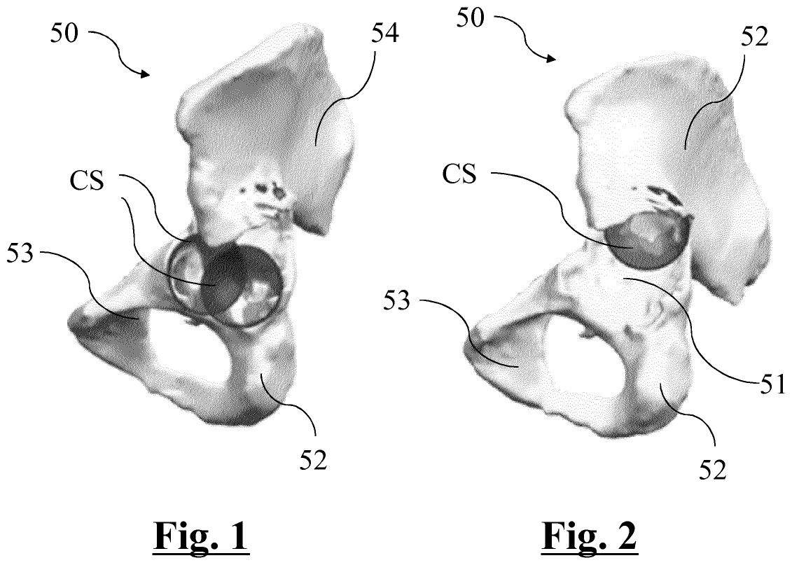

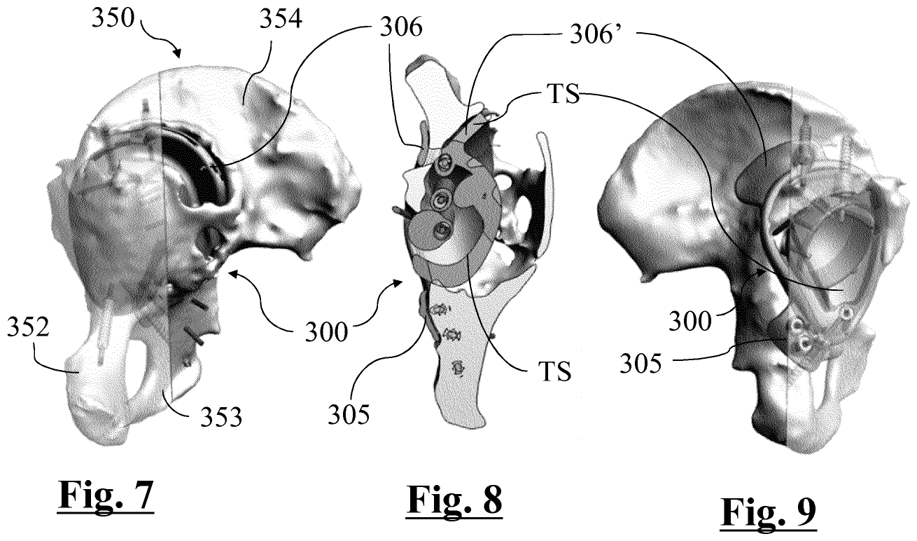

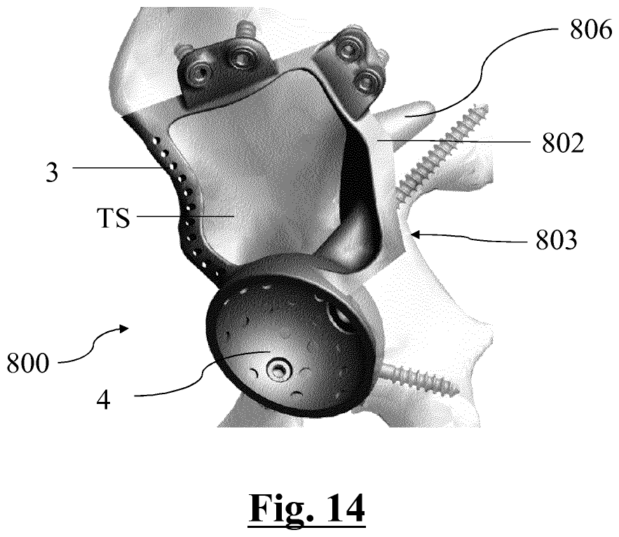

With reference to FIGS. 1-14, hereinafter a series of examples of joint prosthesis component according to the invention will be described, wherein said component is a coxal anchor 10, 100, 200, 300, 400, 500, 600, 700, 800 of hip prosthesis designed to be implanted into a model of coxal bone 50, 150, 250, 350, 450, 550, 650, 750, 850.

With reference to FIGS. 1 and 2, they show a first model of a coxal bone 50 having compromised anatomy which cutting surfaces CS have been overlapped to, the latter simulating a bone tissue removal processing, such as milling, chosen for this particular bone anatomy. In particular, the cutting surfaces CS shown in FIGS. 1 and 2 are hollow half-spheres from which the bone portions to be removed through the processing emerge.

In FIGS. 1 and 2 it is possible to distinguish the various bone elements which make up the coxal bone 50, namely an acetabular cavity 51 placed substantially at the center and below which a ring shape defined by the pubis 52 and the ischium 53 is present. Instead, above the acetabular cavity 51 the ileum 54 extends.

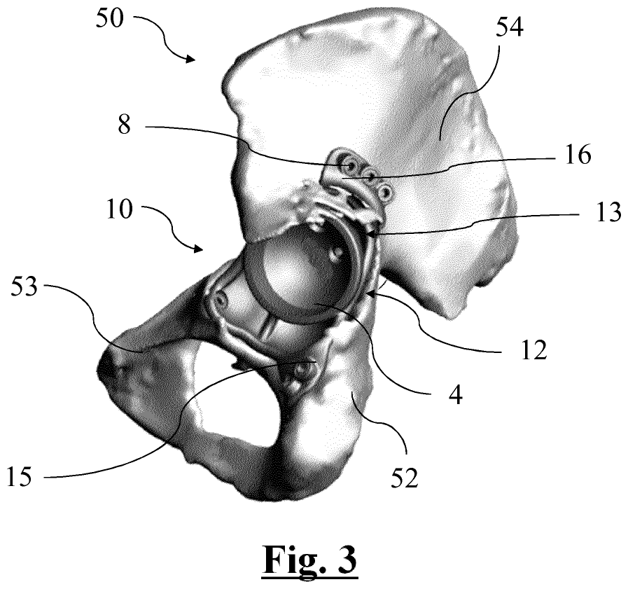

FIG. 3 shows the bone model of FIGS. 1 and 2 as it appears further to the bone removal processing in which a coxal anchor 10 of a hip prosthesis designed according to the present invention is implanted, so as to be customized in order to adapt to the morphology of the coxal bone 50 processed according to what is shown in FIG. 2.

The coxal anchor 10 of FIG. 3 comprises a bone fastening portion made of an acetabular support 12 coupled to the acetabular cavity 51. The acetabular support 12 comprises in turn a distal surface 13 having a morphology suitably made to match with the processed acetabular cavity 51 of FIG. 3 and a concave proximal surface 4 opposite the distal surface 13 adapted to receive the femoral head of a femoral component of hip prosthesis.

As it can be noticed from FIG. 3, the coxal anchor 10 has further additional supports. These additional supports are a pubic support 15 and an iliac support 16 made of flange-shaped appendices extending from the acetabular support 12 and fixed to the pubis 52 and to the ileum 54, respectively, by means of fixing screws 8.

Analogously to the acetabular support 12, the additional supports 15, 16 are customized to adhere to the respective bone element which has been subjected to the previously discussed processing with reference to FIGS. 1-2.

As a skilled person may well understand, the coxal anchor 10 of FIG. 3 is a customized prosthetic component having a morphology defined based on a specific processing of the bone anatomy, so as to maximize the bone-implant contact by filling the bone defects present in order to obtain a suitable stability of the implant and meanwhile to position the prosthetic component, so as to ensure an optimal biomechanics of the prostheses, all this implantable through the as minimally invasive surgical technique as possible.

In the particular case of a coxal anchor, the biomechanics of the prothesis is affected by the positioning of the joint rotation center located at the distal surface of the acromial support and of the orientation in the space of the above distal surface which is defined by the so-called “covering” and “version” of the acetabular support. A suitable position of the rotation center, covering and version, which not necessarily coincide with the anatomic ones, may thus be chosen and optimized in the design step by acting on two variables such as bone processing and prosthesis design.

In other words, unlike the known prosthetic components, the positioning of the component is not conditioned by the need of pursuing implant stability, the morphology of the component may advantageously be defined along with the optimal bone processing, so as to have a suitable positioning and stability of the implant for each patient having compromised anatomy.

The design of the coxal anchor 50 and processing discussed in connection to FIGS. 1-3 is optimized for the specific anatomic model considered. An analogous approach may be repeated for the coxal bone anatomy of each single patient. Hereinafter other examples of coxal anchor designed for other models of compromised coxal anatomy specifically processed will be reported. These examples are indicated as multiple references of one hundred and corresponding elements are indicated with the same tens and units.

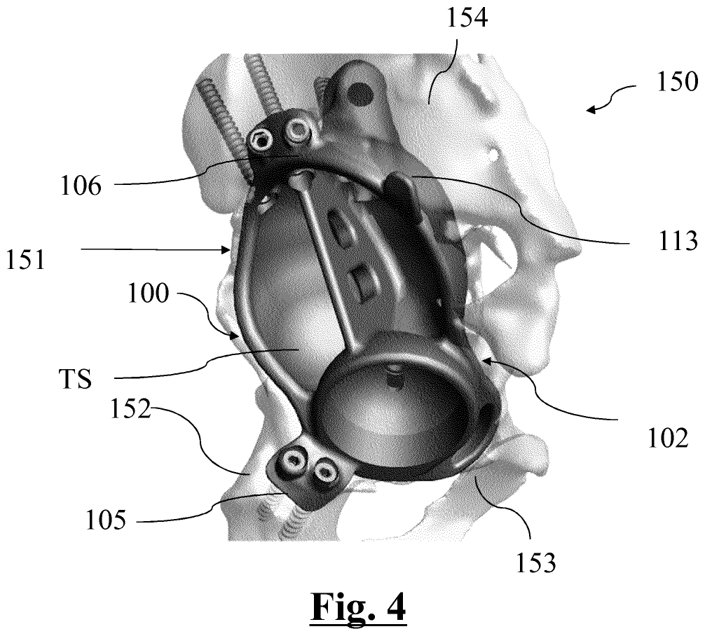

FIG. 4 shows a second model of coxal bone 150 with a more compromised bone anatomy than that of FIGS. 1-3, in which a processing by removal of bone tissue was simulated according to six hemispherical surfaces. FIG. 4 further shows a second example of coxal anchor 100 in which the distal surface 113 of the iliac support 102 is more extensive so as to fill in the bone gaps of considerable size that have dug the acetabular cavity 151 of this specific bone anatomy.

As it can be noticed, the coxal anchor 100 as well has an iliac support 106 and a pubic support 105, both customized so as to adapt to the respective specifically processed bone element.

As a skilled person may well understand, the additional supports 15, 16, 105, 106 used in the coxal anchors 50, 100 of FIGS. 3 and 4 contribute maximizing the contact area between bone and implant, in order to achieve an optimal primary stability.

Beside the two above discussed examples, alternative embodiments of the coxal anchor may provide for one or more pubic supports and/or one or more iliac supports and/or one or more ischial supports according to the basic bone anatomy and to the defined processing.

In addition to the flange shape, these pubic, iliac and ischial supports may take up other appearances; for instance, they may be real projections, extensions or appendices of the distal surface of the acromial support which occupy bone gaps extending within the pubis, ileum or ischium.

The above discussed traditional supports may be made integral with the acetabular support or fixed to the latter in the implantation step by means of cement or other fasteners.

Furthermore, these additional supports may be customized analogously to the acromial supports 12, 102, so as to match with the specifically processed and not processed pubis, ileum or ischium, or be components having standard shape and size adapted to the anatomy.

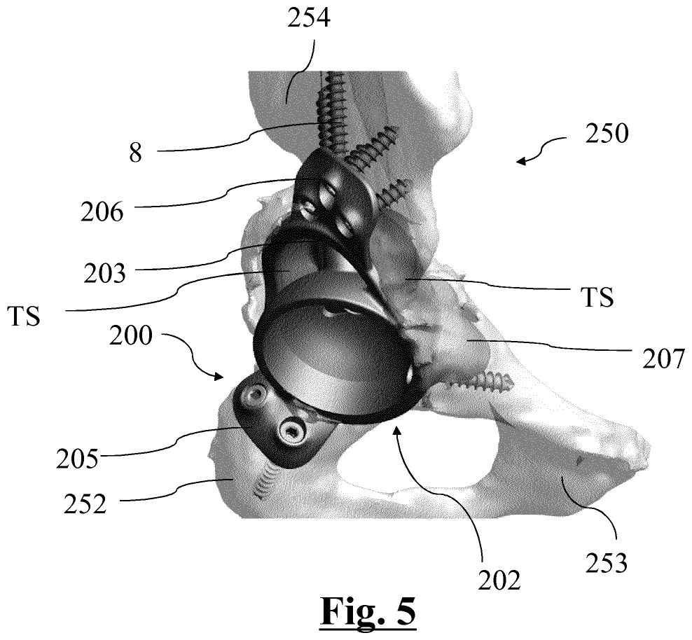

FIG. 5 shows a third example of coxal anchor 200 applied to a third model of specifically processed coxal bone 250 with compromised anatomy comprising, in addition to an acromial support 202, also a pubic support 205 and an iliac support 206 in the shape of flanges fixed to the respective bone element by means of fixing screws 8, in addition to an ischial support 207 in the shape of a projection of the distal surface 203 of the acromial support 202 which fills in a bone gap that invaginates in the ischial bone 253. The above supports 202, 205, 206, 207 are shaped so as perfectly adapt to the bone morphology of the specifically processed coxal bone 250 of FIG. 5, so as to maximize the bone-implant contact area and the stability of the implant with the consequent transfer of loads to the bone uniformly and anatomically.

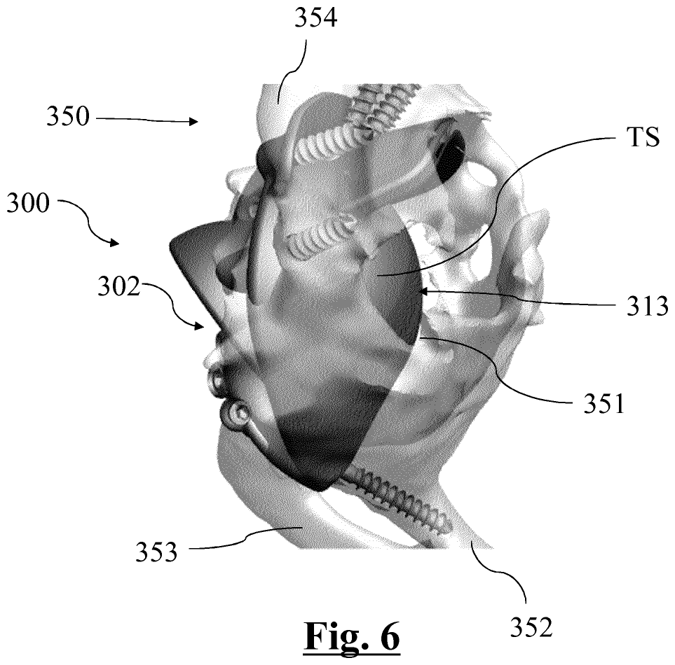

FIGS. 6-9 show a fourth example of coxal anchor 300 applied to a fourth model of a specifically processed coxal bone 350 with a compromised anatomy. The coxal anchor 300, in addition to an acromial support 302 and a pubic support 305 of the flange type, also has two iliac supports 306, 306′ made of a further pair of flanges, a first flange 306 placed at the medial surface of the ileum 354 and a second flange 306′ at the side surface of the ileum 354. As a skilled person will appreciate, the pair of flanges 306, 306′ allows stabilizing the implant against bending or torsional loads.

FIG. 10 shows instead a fifth example of coxal anchor 400 applied to a model of a specifically processed compromised coxal anatomy 450 comprising an iliac support 406 of the stem type made integral with the distal surface 413 of the acromial support 402 and deeply inserted in the ileum 453.

The stem 406 shown in FIG. 10 is of the non-cemented type, having cylindrical shape, tapered end and longitudinal fins. Clearly, alternative embodiments may provide for different shapes of stem, for instance a truncated-cone shape. The stem may further be monolithic or modular, have or not longitudinal fins, comprise at least one portion of porous trabecular material or be coated with hydroxyapatite to favour the primary and secondary fixing to the bone. The stem may be inserted into the bone according to a suitable orientation in order to pursue the best fixing to the bone.

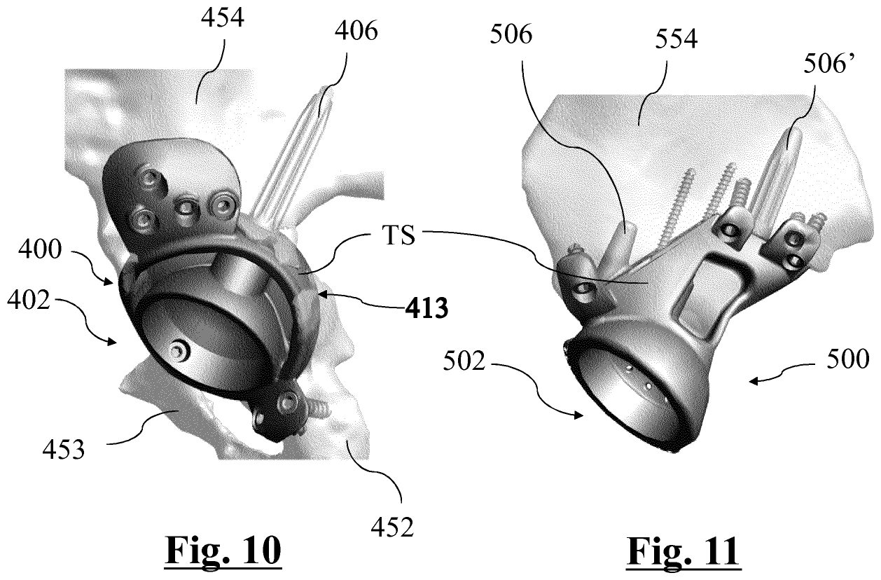

Alternative embodiments may further provide for a plurality of iliac stems having for instance a different morphology, like in the case of the coxal anchor 500 of FIG. 11, in which a stem 506 in the shape of a smooth truncated cone and a cylindrical finned stem 506 are used, as well as three 506″ flange-type iliac supports.

Furthermore, the use of a stem to fix the coxal anchor to other bone elements different from ileum is not excluded.

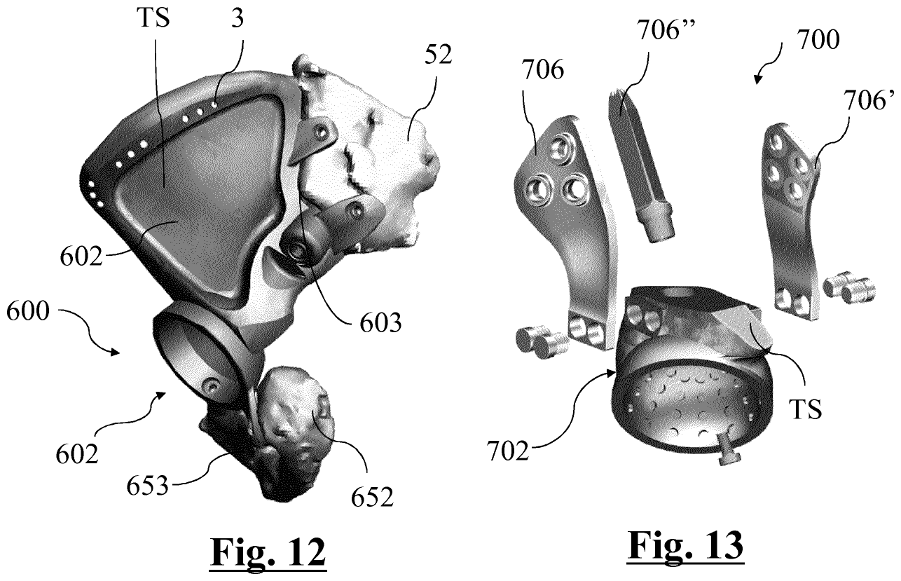

Other three examples of coxal anchor 600, 700, 800 are shown in FIGS. 12, 13 and 14. In particular, the example of FIG. 13 shows two additional supports 706, 706′ fixed to the distal surface of the acromial support 702 and a finned cylindrical stem 706″ of the modular type.

As a skilled person may notice from the figures relating to the above discussed examples of coxal anchor 10, 100, 200, 300, 400, 500, 600, 700, 800, the coxal anchor may at least partially comprise a three-dimensional structure at least partially trabecular porous adapted to be in contact with the bone, thus favouring the primary fixing and integration with the bone. A structure of this type allows the passage of body fluids and reduces bacterial colonization risks of the implant surfaces. In the figures so far cited said trabecular portions are indicated with reference TS.

As it may be noticed for instance from FIGS. 11, 12 and 14, in addition to the parts in trabecular structure TS, the coxal anchor 500, 600, 800 may provide for a plurality of suture holes 3 adapted to allow suturing to the implant the soft tissues resected during the access phase to the implant site once the prosthetic component has been implanted.

As a skilled person may well understand, the use of a trabecular portion and suture holes produce a synergic effect in favouring the integration of the prosthetic implant with the surrounding tissues.

With reference to the example of the previously discussed joint prosthetic component, we hereinafter report a table in which a coxal anchor made according to the invention is compared with the two types of prothesis known nowadays.

| | Fully custom | Standard | Component according |
|---|---|---|---|
| | component | component | to the invention |
| COR | 8-10 | 1-8 | 9-10 |
| Re-positioning | | | |
| Covering | 8-10 | 1-8 | 9-10 |
| optimization | | | |
| Version | 8-10 | 1-8 | 9-10 |
| optimization | | | |
| Anatomical fitting | 6-8 | 1-8 | 7-10 |
| Implant-bone | 6-8 | 1-7 | 7-10 |
| contact surface | | | |
| Bone removal | 8-10 | 1-6 | 5-10 |
| Stability | 4-10 | 1-8 | 5-10 |

The above reported table shows a range 1-10 in which 10 is the best for each evaluation parameter of an implant goodness. From the table a skilled person may immediately appreciate how a coxal anchor made according to the invention allows achieving an excellent result in all the evaluated parameters, namely optimizing for instance both positioning, fitting and stability of the implant.

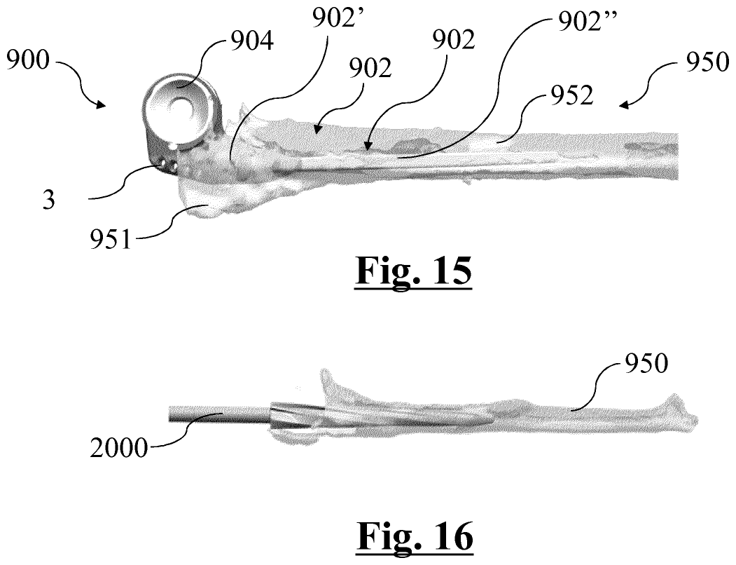

With reference to FIGS. 15-22, a series of examples of joint prosthesis component according to the invention will now be described in detail, in which said component is an ulnar component 900, 1000, 1100 of hip prosthesis designed to be implanted in a model of ulna 950, 1050 of a patient with compromised bone anatomy of the elbow. In particular, said ulnar component may advantageously be used in ulnas with high bone loss in the proximal area, for instance up to half the length of the bone.

As it will be immediately apparent to a skilled person, the ulnar components hereinafter discussed are usable in a total knee prosthesis of the hinged type. This type of elbow prothesis provides for an ulnar component fixed to the proximal end of the ulna and a humeral component fixed to the distal end of the homer, which are hinged to each other thus allowing the rotation about a pin.

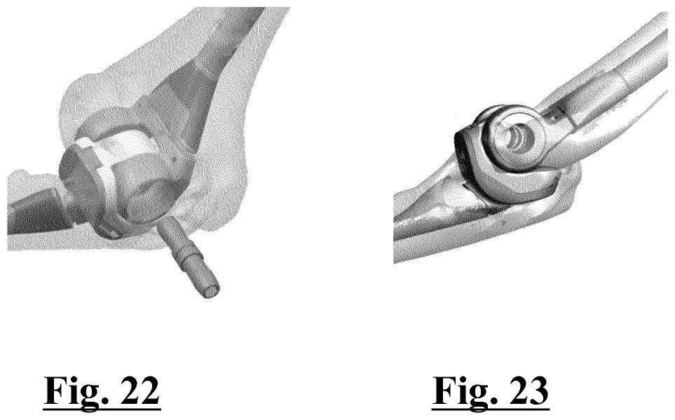

This obviously does not exclude for an ulnar component of other types of knee protheses—for instance not hinged or partial ones—or other components of knee prostheses to be made according to the invention. FIGS. 22 and 23 show total elbow protheses according to the prior art, of the hinged and not hinged type, respectively.

In FIG. 15 the ulnar component 900 is shown being inserted at the proximal end of the anatomic model of an ulna 950 with compromised anatomy comprising a proximal epiphysis 951 and a diaphysis 952, both specifically processed.

Said ulnar component 900 comprises in turn a joint portion 904 adapted to be hinged to a corresponding portion of a humeral component of knee prothesis and a bone fastening portion 902 in which a contact surface 903 is defined, which is adapted to be in contact with the bone when the component is implanted as shown in FIG. 15.

The above fastening portion 902 has an under-fastening proximal portion 902′ and a distal under-fastening portion 902″. The proximal 902′ and distal 902″ fastening portions are advantageously shaped to adapt to the morphology of the proximal epiphysis 951 and of the diaphysis 952 which are specifically processed by means of at least one surgical instrumentation 2000.

As it may be noticed in FIG. 15, the fixing portions 902′, 902″ are fixed to the bone without cement, however this does not exclude the use of cement in alternative embodiments.

Furthermore, in alternative embodiments just one of the two portions 902′, 902″ may be customized in connection to a particular processing of the bone and the other one may be customized to the not processed or standard bone anatomy.

Analogously to what has been discussed in the previous examples of joint prosthesis component, the ulnar component 900 as well is designed considering two variables such as component morphology and bone processing, so as to conceive a prosthetic component which can produce optimal stability and biomechanics of the prothesis once implanted.

As it will be clearer hereinafter, the discussed ulnar components allow minimizing the bone to be removed, gaining stability and integration of the implant to the bone and to the soft tissues and obtaining a correct joint movement.

The processing of the ulna 950 may advantageously be performed by means of a customized instrumentation, in order to obtain the desired processing. FIG. 16 shows an example of customized surgical instrumentation 2000 while performing the preparation processing of the intramedullary canal of the diaphysis 952 in order to receive the distal fastening portion 902″.

Obviously this does not exclude for the bone to be processed by means of standard instruments.

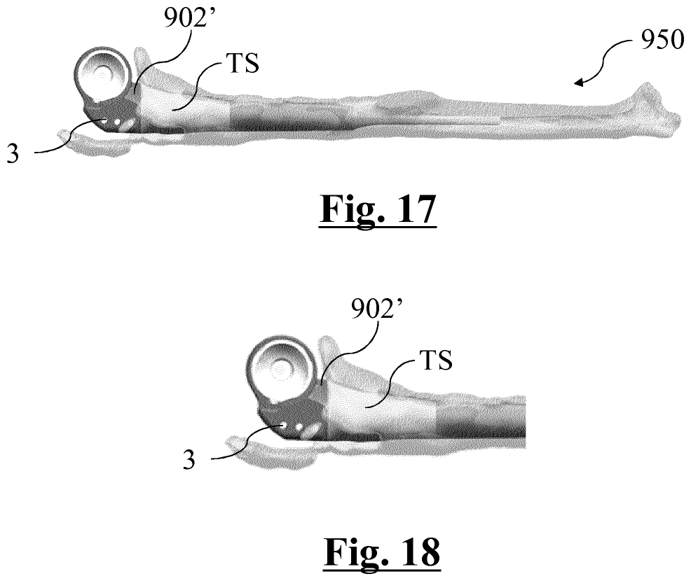

As it is clear from FIGS. 17 and 18, the ulnar component 950 has a fastening portion 902′ which includes a trabecular three-dimensional porous structure TS and suture holes 3 analogously to the previous examples. As also previously discussed, the latter features allow increasing the stability of the component and the implant-bone contact. Obviously this does not exclude for the trabecular structure and the suture holes not to be present or located in other parts of the component.

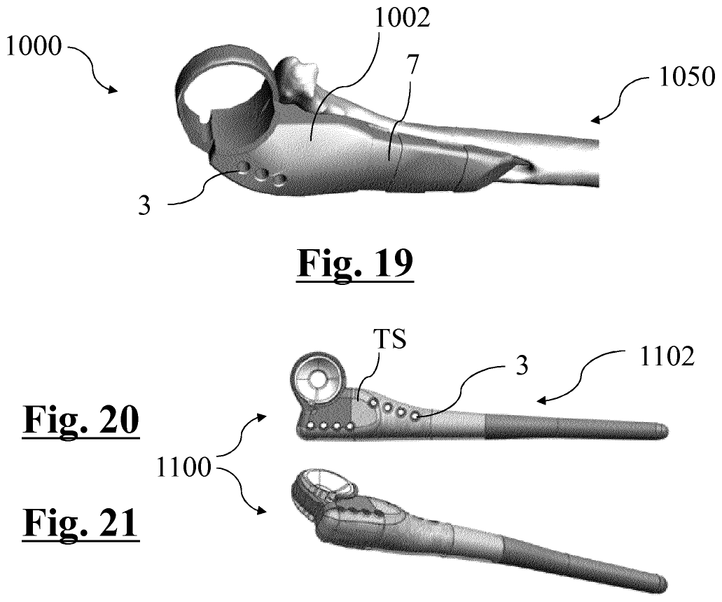

FIG. 19 shows another example of ulnar component 1000 with suture holes 3 and grooves 7 also introduced to increase the implant stability.

FIGS. 20 and 21 show a further example of ulnar component 1100 comprising a distal fastening portion 1102′ having a trabecular portion TS in which suture holes 3, suitably positioned in an area such that the brachial muscle can be sutured to the implant, are formed. Other suture holes 3 are formed in a non-trabecular portion which is more proximal than the previous trabecular portion, so as to suture the triceps muscle thereto.

Even in this case the suture holes 3 and the trabecular portion TS perform a synergic action in ensuring a suitable integration and stability of the implant.

Like the morphology of the component, the number and position of the suture holes 3 and trabecular portions TS may also be customized according to the bone anatomy of the patient and to the chosen bone processing.

1. A joint prosthesis component adapted to be fixed to a first bone extremity of a joint of a single patient having compromised anatomy, wherein said first bone extremity is a coxal bone of a patient with compromised anatomy and said joint prosthesis component is a coxal anchor of hip prosthesis;
said joint prosthesis component comprising:
at least one fastening portion adapted to be in contact with said coxal bone;
at least one joint portion adapted to be directly coupled with a second bone extremity of said joint or with a conjugate second joint prosthesis component, in turn fixed to said second bone extremity, said second bone extremity being a proximal end of femur of a patient and said second prosthesis component being a femoral component of hip prosthesis;
wherein said at least one fastening portion comprises an acetabular support having a distal surface adapted to be at least partially in contact with an acetabular cavity of said coxal bone and a concave proximal surface opposite said distal surface adapted to receive a head of said femoral component of hip prosthesis,
wherein said fastening portion at least partially comprises a porous trabecular three-dimensional structure having a contact surface adapted to be in contact with the bone, promoting primary fixation and integration with the bone,
wherein said fastening portion is at least partially customized to be specifically shaped with respect to the morphology of said first bone extremity processed by means of at least one surgical instrument,
wherein said distal surface is counter-shaped with respect to said acetabular cavity processed by means of said at least one surgical instrument,
and wherein said distal surface is a customized portion which adapts to the morphology of the acetabular cavity of the patient with a specifically processed compromised anatomy.

2. The joint prosthesis component according to claim 1, wherein said fastening portion further comprises at least one suture hole to restore the continuity with soft tissues at the end of the implantation of said joint prosthesis component.

3. The joint prosthesis component according to claim 2, wherein said contact surface of said three-dimensional structure and/or said at least one suture hole are customized according to the compromised joint anatomy.

4. The joint prosthesis component according to claim 3, wherein said fastening portion further comprises at least one iliac support adapted to abut at an ileum of said coxal bone.

5. The joint prosthesis component according to claim 4, wherein said at least one iliac support is shaped to at least partially match with said ileum of said coxal bone processed by means of at least one surgical instrument.

6. The joint prosthesis component according to claim 4, wherein said iliac support consists of at least one stem which extends from said distal surface of said acetabular support and is deeply inserted into said ileum.

7. The joint prosthesis component according to claim 4, wherein said iliac support consists of at least one medial flange or lateral flange.

8. The joint prosthesis component according to claim 1, wherein said fastening portion further comprises at least one pubic support adapted to abut at a pubis of said coxal bone.

9. The joint prosthesis component according to claim 8, wherein said at least one pubic support is shaped to at least partially match with said pubis of said coxal bone processed by means of at least one surgical instrument.

10. The joint prosthesis component according to claim 1, wherein said fastening portion further comprises at least one ischial support adapted to abut at an ischium of said coxal bone.

11. The joint prosthesis component according to claim 10, wherein said at least one ischial support is shaped to at least partially with match said ischium of said coxal bone processed by means of at least one surgical instrument.
